(19) United States
(12) Patent Application Publication
(10) Pub. No.: US 2024/0319126 A1
(43) Pub. Date: Sep. 26, 2024

(54) MULTI-SCALE, NANO- TO MESOSTRUCTURAL ENGINEERING OF SILK BIOPOLYMER-INTERLAYER BIOSENSORS FOR CONTINUOUS CO-MONITORING OF NUTRIENTS IN FOOD

(71) Applicant: The Regents of the University of California, Oakland, CA (US)

(72) Inventors: Peter Tseng, Irvine, CA (US); Manik Dautta, Irvine, CA (US); Kazi Khurshidi Haque Dia, Irvine, CA (US); Amirhossein Hajiaghajani, Irvine, CA (US)

(73) Assignee: The Regents of the University of California, Oakland, CA (US)

(21) Appl. No.: 18/579,317

(22) PCT Filed: May 13, 2022
(86) PCT No.: PCT/US2022/029294

§ 371 Date: Jan. 12, 2024

Related U.S. Application Data
(60) Provisional application No. 63/222,898, filed on Jul. 16, 2021.

(51) Int. Cl. G01N 27/22; G01N 33/02

(52) U.S. Cl. CPC G01N 27/221; G01N 27/227; G01N 33/02

(57) ABSTRACT

The present embodiments relate to the multi-scale engineering of silk biopolymer-interlayer sensors for co-monitoring of nutrients. By manipulating various nano- to meso-structural properties of such biosensors, obtained are sensors with programmable sensitivity and selectivity to salts, sugars, and oils/fats. Notably, this approach requires no specialized nanomaterials or delicate biomolecules. Programmable biosensors are further formatted for wireless readout, and characteristics of these passive, wireless nutrient monitors are studied in-vitro. It is anticipated that such sensors can be utilized in emerging dictary tools for applications across food tracking and human health. In addition, it is expected that such strategies in structural engineering of sensors can be adaptable to existing or emerging selective or partially-selective sensors.

### CROSS-REFERENCE TO RELATED APPLICATIONS

The present application claims priority to U.S. Provisional Patent Application No. 63/222,898 filed Jul. 16, 2021, the contents of which are incorporated herein by reference in its entirety.

### STATEMENT OF GOVERNMENT SUPPORT

This invention was made with Government support under Grant No. R21CA239249, awarded by the National Institutes of Health (NIH). The Government has certain rights in the invention.

### TECHNICAL FIELD

The present embodiments relate generally to food/nutrition sensing, and more particularly to nano- to meso-scale engineering of biopolymer sensors configured to co-readout major nutrient direct from food (salt, sugar, fats).

### BACKGROUND

Food nutrients are typically extracted via fractionation and mass spectroscopy. This is not suitable for on-the-fly nutrient monitoring. Silk biopolymer sensors were previously demonstrated. However, these sensors respond very slowly, and are only able to monitor salt/sugar. These were not demonstrated to be responsive to read foods.

It is against this backdrop that the present Applicant sought to advance the state of the art.

### SUMMARY

In one or more embodiments, programmable, silk biopolymer-based, wireless and passive biosensors are provided for discrimination and co-readout of nutrients direct from real food. This is enabled by nano- to meso-structural engineering of the sensing construct. This approaches requires no specialized nanomaterials or delicate biomolecules, while enabling robust biosensors with programmable sensitivity and selectivity to salts, sugars, and oils/fats.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1 illustrates aspects of Biopolymer-interlayer sensors for nutrient monitoring. In FIG. 1, sub-figure (a) is Schematic presentation of a parallel plate lossy capacitor where two metal electrodes are interceded by a functional biopolymer—this is modeled as a parallel RC. Sub-figure (b) illustrates Change in imaginary permittivity, interlayer thickness, and real permittivity due to infiltration of salt, glucose, and fat, respectively. Biopolymer possesses a solid, water-pinning phase that interacts with water-soluble nutrients, whereas pores in the polymer are an engineerable open phase that responds to oil/fat. Sub-figure (c) ukkystrates Transformed parallel plate sensor into a broad-side coupled, split-ring resonator architecture for wireless readout/characterization. This is represented as a simple RLC circuit that can be read out remotely via inductive coupling through an antenna. Sub-figure (d) illustrates Multilayer device structure where the capping, metal and interlayer are individually engineered to modulate sensor selectivity, sensitivity, and response time.

FIG. 2 illustrates In-vitro characterization of the sensors with structural porosity engineered at various layers of the biopolymer and metal electrode. In FIG. 2, sub-figures (a), (b), (c), (d), and (e) are Schematics of device construct possessing various combinations of layer porosity. Generally, higher lettering corresponds to more layers possessing porosity. Alongside the schematics are the temporal response of the sensor to various nutrients, simulated in-vitro as sodium chloride, glucose, and oleic acid. Generally, the interlayer pororsity controls both the sensitivity and response time of the sensor, whereas capping and electrode layer porosity impacts the response time.

FIG. 3 illustrates aspects of In-vitro characterization of device response to various nano- to meso-scale structural modifications. In FIG. 3, sub-figure (a) illustrates Effect of silk fibroin crystallization process. i) schematic of the film crystallization methods (methanol treatment-MT and water-anneal-WA), ii) magnitude response of a NP_CIM sensor in salt, iii) frequency response of a NP_I_P_CM sensor in glucose, and iv) frequency response of a P_CIM sensor in oleic acid. Sub-figure (b) illustrates Impact of capping layer removal. i) schematic of the structural modification (with Cap and without Cap). ii), iii), and iv) Response of relevant sensors to step changes in salt, glucose, and oleic acid concentration. Sub-figure (c) illustrates Impact of the interlayer thickness. i) schematic of the variable interlayer thickness. ii), iii), and iv) Response of relevant sensors to step changes in salt, glucose, and oleic acid concentration.

FIG. 4 illustrates aspects of Optimized sensor performance in complex fluidic environments. In FIG. 4, sub-figure (a) illustrates Structural parameters for the salt-optimized (SaO), sugar optimized (SuO), and fat optimized (FO) sensors. Sub-figure (b) illustrates Magnitude response of a SaO sensor in increasing concentration of salt. Sub-figure (c) illustrates Frequency response of a SuO sensor in increasing glucose concentration. Sub-figure (d) illustrates frequency response of a FO sensor in increasing oleic acid volume. Sub-figure (e) illustrates Magnitude (i, ii) and frequency (iii, iv) response of a SaO and a SuO sensor in a salt and glucose mixture at body temperature. Sub-figure (f) illustrates Magnitude (i, ii) and frequency (iii, iv) response of a SaO and a FO sensor in salt and subsequently oleic acid at body temperature.

FIG. 5 illustrates Nutrient discrimination and co-readout from real foods. The bottom sub-figure illustrates how Shift in frequency maps to the sugar and/or fat content of the food, whereas magnitude response maps to the salt content of the food. Labels of S, F, and IS refer to the sugar, fat, and ionic strength of the food in units of g/100 mL, g/g, and charge/L respectively. This is extracted from the nutrient data provided by the food manufacturer. In FIG. 5, sub-figure (a) illustrates Response of a SuO and a FO sensor in milks (fat free H&H, low-fat chocolate with added sugar, chocolate, and vitaminD whole milk). Sub-figure (b) Response of a FO sensor in solid foods (extra lean turkey breast and fatty beef). Sub-figure (c) illustrates Response of a SuO and a FO sensor in various tea drinks (green tea, sugar added green tea and sugar added milk tea). Sub-figure (d) illustrates Response of a SaO and a FO sensor in broths and soups (unsalted chicken broth, low salt/fat vegetable broth, salty/fatty chicken stock and butter added salty chicken stock).

Supplementary Figures S1-S8 illustrate additional example aspects of the present embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

The present Applicant recognizes, among other things, that nutrient sensing is core to human health. There is no tool that can currently resolve the major nutrients of food. The present embodiments relate to an approach that includes both multi-scale engineering of sensors, as well as arraying them that enables co-readout of salt/sugar/fat. This has been utilized to measure these nutrients directly from real foods continuously. This type of co-readout from real solid/liquid foods has not been demonstrated.

### INTRODUCTION

Nutrition and diet play a critical role in the maintenance of the body, with a healthy diet associated with broad, positive markers of human health including—but not limited to—reduced risk and improved maintenance of disease, improved mood, reduced inflammation, improved physical performance, increased muscle mass, and more [1-5]. During food consumption, it is the nutrients that we consume (water, salt, fats, and protein) that sustain daily functions such as providing energy, growing tissue, and maintaining cellular function [6-8]. Despite its importance in health and human well-being, there are currently no tools that can monitor the nutrition content of foods. Current approaches to measure the nutrient content of food largely occur by fractionation and subsequent mass spectroscopy [9-13]. While these methods are accurate and effective, these require significant sample preparation and expensive/large machinery, and are thus not suitable for measuring nutrient content direct from foods in on-the-fly settings so critical to many emerging applications.

The major nutrients of food include carbohydrates (separated into complex carbohydrates and simple sugars), salts, oils or fats, protein, and finally water itself. These compose the majority of the weight of food, with carbohydrates composing 5-10%, fats or oils at 5 to 30%, salts at up to 1%, and proteins at up to 10 percent weight (with water composing the remaining mass and micronutrients at small concentrations) [14,15]. Complex carbohydrates and proteins are difficult (if not impossible) to quantify direct from foods because these are not broken down significantly before intake. However, core nutrients such as simple sugars, salts, oils/fats, and water (that exist in molecules close to their metabolized forms) are key potential targets for nutrient sensing platforms. Indeed, the under-consumption/over-consumption of these critical nutrients are linked to a variety of markers of human health including mental state [16-18], disease onset/maintenance [19,20], and body development [21-23].

The present Applicant recently proposed and demonstrated a partially-selective biosensor composed of a silk fibroin biopolymer-interlayer interceding two electrodes (structured into a wireless format) for nutrient detection [24]. The silk biopolymer forms a dense, water-absorbent membrane that inherently performs sample preparation on the fluidic environment, while absorbing and/or swelling in response to salts or simple carbohydrates. This device was attached to a human tooth and used to measure the sugar, salt, and alcohol content from a variety of oral liquids. This sensor had a variety of limitations including relatively slow response times, lack of programmability in sensor performance, and no ability to measure oils or fats (a key nutrient in food). The present embodiments utilize nano- to meso-scale engineering of these biosensors to enable programmable biosensors optimized for detection of salt, sugars, and fats from foods. Key to this strategy is the use of basic structural modifications of the biopolymer and metal electrode to modulate sensor performance. This strategy stands in contrast to many modern strategies that utilize inaccessible nanomaterials [25-29] and delicate bio-molecular recognition elements [30-33]. As a proof of concept, it is demonstrated that pairs of sensors can be used to discriminate between and co-monitor simple sugars, salts, and oil/fat direct from a variety of foods. It is expected these biopolymer-based dielectric sensors to be robust in a variety of environments and scenarios, while exhibiting programmability to meet the needs of emerging applications related to food storage, preparation, and intake. For example, these biocompatible, modular sensors may be ideal in wearable oral appliances to monitor complex nutrients from food during intake. Such appliances have previously been used to monitor salt, urea, or motion within the mouth [34,35].

### Results and Discussion

Silk fibroin, biopolymer-interlayer sensors

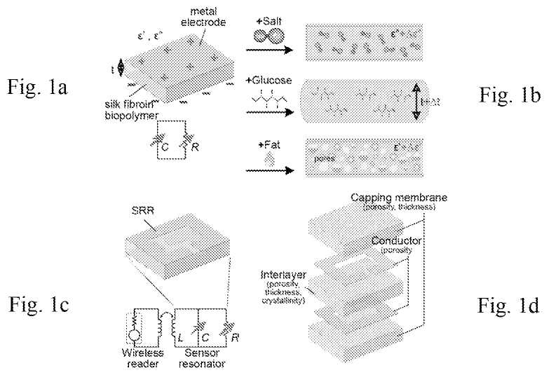

The base sensing element is composed of engineered silk fibroin biopolymer interceding two metal electrodes forming a capacitor. The response of this structure to biofluid can be described using complex impedance. The biopolymer interlayer acts as a lossy dielectric material and can be represented as a parallel combination of a capacitor and a resistor (FIG. 1a). This capacitance and resistance vary with the changing physical and dielectric properties of the interlayer in response to nutrients. In this study, selectively introduced are open pores into silk biopolymer to modulate its selectivity. Thus, the biopolymer interlayer is composed of a solid phase (hygroscopic, water-pinning silk fibroin), and open phase (or pores within the solid protein film) shown in FIG. 1b. The solid phase interacts only with salt and sugar due to the diffusion of these molecules through the water-carrying silk fibroin (these are also the most prevalent small molecules in food). For salt, the solid phase will absorb the charged ions and the dielectric will exhibit a large change in the in ε″ or loss of the interlayer. For sugars it will absorb simple carbohydrates (these need to be small-chained such as lactose/sucrose/fructose/glucose), which induces swelling in the protein, thereby changing the thickness, t. These two primarily affect either the real (sugar) or imaginary (salt) portion of the capacitance, and thus each nutrient class can be discriminated from each other readily during measurement. The porous section irreversibly absorbs fat from the environment (i.e. the holes will fill with oil/fat over time, replacing water). This is reflected in an irreversible continual shift in &′ of the sensor when exposed to oil/fat, because oil/fat has a significantly lower permittivity than water. Thus oil/fat signal primarily overlaps in readout with the sugar measurement (by impacting the real component of capacitance). For the base unmodified sensor, however, there are very few pores in the spun-coat biopolymer, and thus these base sensors present very low sensitivity to fatty acids and almost no sensitivity to triglycerides. It is believed that this ability to co-monitor both oil/fats and water-soluble nutrients may be unique to silk fibroin polymer, which exhibits both hydrophobic and hydrophilic regions [36,37]. Effectively, these biosensors are partially-selective sensors whose selectivity is modulated with nano- to mesostructural engineering. This strategy alongside proper data extraction can be used to discriminate various major nutrients in food.

This sensor is finally built into a broadside-coupled split ring resonator (bcSRR) format [38-41] and interrogated wirelessly as shown in FIG. 1c. The fundamental response of this wireless sensor can be modelled using a simple series RLC equivalent circuit. The changes in capacitance and resistance are reflected in the spectral response of the resonator, which is read out wirelessly via inductive coupling to a Vector Network Analyzer (VNA). Changes in the effective resistance modulates the signal magnitude (salt will significantly reduce the magnitude), while changes in the effective capacitance shifts the resonance frequency of the sensor (sugar and fat/oil both increase the resonant frequency). The final sensor construct is shown in FIG. 1d, and is composed of capping biopolymer membranes surrounding the central electrode-biopolymer interlayer-electrode stack. Capping membranes enhance structural stability and improve the specificity of the sensor. Herein is studied how the engineering of the thickness, porosity, and/or crystallinity of the silk-interlayer biosensor structural layers can be used to tune sensor selectivity, sensitivity, and response time to nutrients.

### Nano- to Meso-Scale, Structural Engineering of Biopolymer-Based Sensors.

A first study focuses around the impact of structural layer porosity on the response of sensors to nutrients (sodium chloride, glucose, and oleic acid fatty acid for in-vitro studies). Here, the primary aim is to enhance sensor response speed. To simplify these experiments, the thickness of the various structural layers were held constant (5 μm capping, 3.5 μm interlayer). Pores in the metal electrodes were generated alongside the formation of our split-ring coils using a vinyl-cutter (250×250 μm² holes separated by 500 μm). Highly-porous silk biopolymer is generated by drying the biopolymer alongside polyethylene glycol (PEG) polymer (15% weight of PEG in the final formulation) where the PEG is subsequently leached out of the biopolymer to yield a porous silk biopolymer (a photo of the sensor and the SEM images of the nonporous and porous silk fibroin are shown in Suppl. Figure S1). The present disclosure has introduced abbreviations to refer to the construction of various sensors studied, where NP refers to nonporous, and P refers to porous, whereas for the layers C refers to capping, I refers to interlayer, and M refers to the metal electrode. For example, NP_CIM refers to a nonporous capping, inter-, and metal layers. NP_I_P_CM would refer to nonporous interlayer, and porous capping and metal layers.

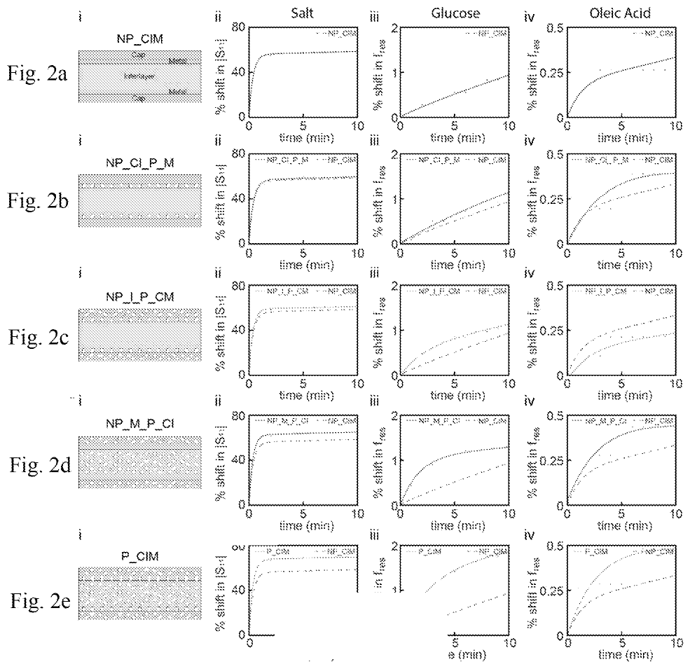

The response of the basic all non-porous biopolymer-interlayer sensors (NP_CIM, similar to as previously published [24]) to a set concentration of salt, glucose, and oleic acid is presented in FIG. 2a. The raw spectral response of the sensor to given concentrations of nutrients is given in Suppl. Figure S2 and illustrates that salt impacts the magnitude of the sensor and not the frequency, while sugar impacts primarily the frequency of the sensor and not the magnitude (sugar does lead to a minor increase in the magnitude however this effect is much smaller than magnitude shifts generated by relevant concentrations of salt). While these base sensors demonstrate a fast response to salt (˜52% in a minute), these sensors exhibited a relatively slow response to glucose (1% frequency shift over 10 minutes), and a slow/weak response to the fatty acid (0.25% frequency shift in 10 minutes). This slow/weak response to fatty acid is attributed to the low porosity of this basic biopolymer sensor. First systematically enhanced was the porosity of various layers either on their own or in combination (FIG. 2b-e). Meso-scale engineering of holes in the electrode facilitates transport of nutrients past the impermeable metal, whereas enhancing the porosity of the biopolymer itself increases nutrient diffusivity through the biopolymer film. The responses of these modified sensors are then compared with the base non-porous sensor (NP_CIM). As expected, introduction of porosity in the electrode, and then enhancement of porosity in the biopolymer capping and interlayer improves the response time and eventually the sensitivity of these sensors to various nutrients. IT was found the porosity/form factor of the interlayer itself controls the sensitivity of the sensor. Retaining a solid interlayer while introducing porosity in the capping and electrode layers around this region enhances the response time of the sensor but not necessarily its sensitivity (FIG. 2b-c). However, increasing the porosity of the interlayer itself led to both increases in the speed of sensor response, as well as sensor sensitivity to all nutrients (FIG. 2d-e). In sum, increasing porosity broadly led to multi-fold increases in response time of the sensor, while increasing porosity in the interlayer led to increases in sensitivity of around 30 to 100% depending on the nutrient. One interesting point, however, is that increasing the porosity in the capping and electrode layers while retaining a solid interlayer led to a unique reduction in sensor response time to oleic acid. It is believed this is due to the unique interactions that occur in the sensor, where oil in the larger pores of the capping layer act to reduce the energetic favorability of water transport away from within the interlayer.

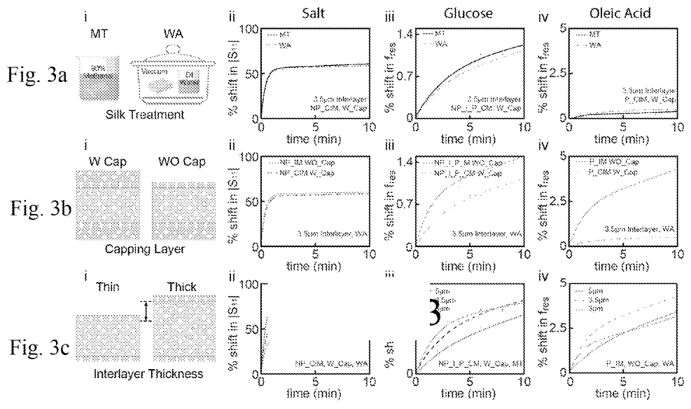

Next studied were changes in sensor performance due to a variety of additional structural modifications. This includes silk fibroin crosslinking/crystallization technique, the presence/removal of the capping membrane, and changes in the interlayer thickness. Silk fibroin can be physically crosslinked using a variety of methods; typically, either water annealing or methanol treatment is used to transform silk from amorphous to crystalline state, which stabilizes the biopolymer film in water. It was found that methanol treated sensors (as compared to water-annealed sensors) exhibited a slightly faster response speed to glucose, however little to no change in sensitivity to salt/oleic acid (FIG. 3a). Next was studied the impact of removing the capping layer from the sensor. This created a minor increase in salt response speed and generated a moderate enhancement in the sugar response speed—as expected the removal of the capping membrane improves transport of nutrients into the interlayer. However, it was additionally found this removal had a dramatic effect on the sensor selectivity, switching the sensor to a fat/oil-dominated response. Without a capping membrane, hydrophobic oils readily and rapidly absorb into the open pores of the structure. This replaces water in the openings and leads to a rapid and large shift in the resonant frequency of the sensor (FIG. 3biv). The 10-minute oleic acid response of these particular sensors led to an almost 5% shift in the resonant frequency, far more sensitive than the sugar response. Finally, studied was the impact that changing the interlayer thickness on sensor response to various nutrients. Thicker interlayers significantly increased sensor sensitivity to salt—it is believed that this is because such thicker layers are more readily able to absorb high concentrations of salt into the interlayer. Thinner layers, however, led to more rapid sugar response times. It is believed that this is because of the swelling response of the polymer to sugars, where thinner interlayers are able to swell more rapidly than thicker layers due to lower structural inertia (this response is in line with swelling hydrogels that have been previously investigated [39]). Unlike sensor response to salt and sugar—which exhibited a consistent trend due to changing thickness—this was not the case for the uncapped, porous interlayer sensor response due to oleic acid. Again, it is believed this is likely due to unexpected interactions between the porous biopolymer, interlayer water, and oil that are not well understood at this moment. Additional related studies on the impact of structural modification on sensor response is given in Suppl. Figure S3 and Suppl. Figure S4.

### Optimized Biopolymer Sensors for Enhanced Sensitivity/Selectivity to Salt, Sugar, and Oil/Fat

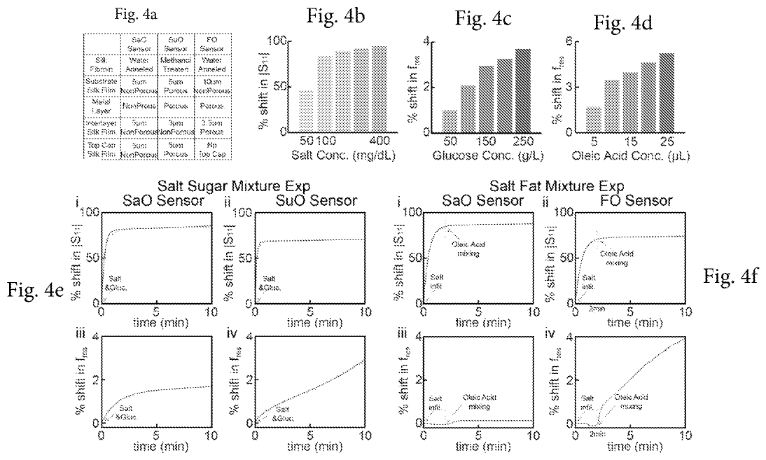

While it is anticipated that additional optimization is possible to further enhance sensor behavior, three sensors designs were settled on to optimized to salt, sugar, and oil/fat. For salt, selected was a 5 μm interlayer, water-annealed construct that exhibited no porosity in any layer. For sugar, chosen was a 3 μm non porous interlayer, methanol-treated construct that possessed porous electrode and capping layer. This construction strategy reduces each sensor's sensitivity to oils/fats, while possessing structural tweaks that enhance sensor response speed or sensitivity to the relevant nutrient. Notably, both these sensors can co-monitor salt and sugar content, however exhibit tweaks to tune particular sensors for optimal measurement of salt and sugar respectively. Thirdly a 3.5 μm interlayer, water-annealed uncapped construct with all layers being porous was chosen for oil/fat (FIG. 4a). The sensitivity of respective sensors to concentrations relevant to food is shown in FIG. 4b-d. A duplicate study was performed comparing the temporal response of these three respective sensors to 100 g/L glucose, 100 mg/dl NaCl, and oleic acid. These results validated this design approach, with salt sensors exhibiting enhanced response to salt, but moderate and weak response to glucose and oleic acid. Sugar sensors exhibiting an enhanced response to sugar, yet moderate and weak response to salt and oleic acid. While fat sensors possessed an enhanced response to oleic acid, as expected from our optimization studies (Suppl. Figure S5).

The response of the sensors can be measured immediately, with the sensor response rate proportional to the nutrient differential, and the saturated measurement proportional to the total nutrient concentration (Suppl. Figure S6). While the saturated measurement takes less than a minute for salt sensing, it can take upwards of 10 minutes for sugar sensing. This means that analytical methods must be employed to determine the nutrient concentration in volatile environments, where the rate of sensor signal change will play an important, determining role in extracting the sugar concentration. Additionally studied was sensor response in elevated temperature (˜38° C., temperature of a body), and it was found that the sensors respond faster in elevated temperature (Suppl. Figure S7). A full reversibility study was performed, demonstrating that both salt and glucose sensors reset to their initial state due to salt/glucose (at either various relevant exposure times), however the fat sensor exhibits irreversibility (Suppl. Figure S8), as the fat/oil is trapped within the pores of the silk fibroin polymer. This means that fat sensing exhibits a limited lifetime, however in exchange these sensors possess memory of oil/fat that can be measured at any point in time and indicate the previous exposure of the sensor to oil/fat (i.e. these do not have to be continuously monitored).

Discrimination and Co-Readout of Salts, Sugars, and Fats Direct from Liquid to Solid Foods

A final in-vitro study was performed, testing sensor response to mixtures of nutrients at body temperature. First, salt-optimized (SaO) and sugar-optimized (SuO) sensors were tested in a mixture of 100 mg/dL NaCl and 100 g/L glucose (FIG. 4e). As expected, because these two nutrients map primarily to either the magnitude (salt) or frequency shift (sugar) of the sensor, a combination of the two nutrients will yield a shift to both magnitude and resonant frequency of the sensor. Next a SaO and a fat-optimized (FO) sensor were tested in a pseudo-mixture of 100 mg/dL salt and 100% Oleic acid (these two are immiscible so it is not a single solution). The sensors were exposed to the salt solution only first, and then oleic acid was added and mixed so as to present the oil to the sensor (FIG. 4f). As expected, the salt has little effect on the resonant frequency shift of the sensor, while significantly modulating the magnitude response. Upon introduction of oleic acid above the sensor, it exhibits a rapid shift in resonance frequency, while not impacting the salt response. These experiments confirm the solid (water-responsive) and open (oil/fat-responsive) facets of the sensor.

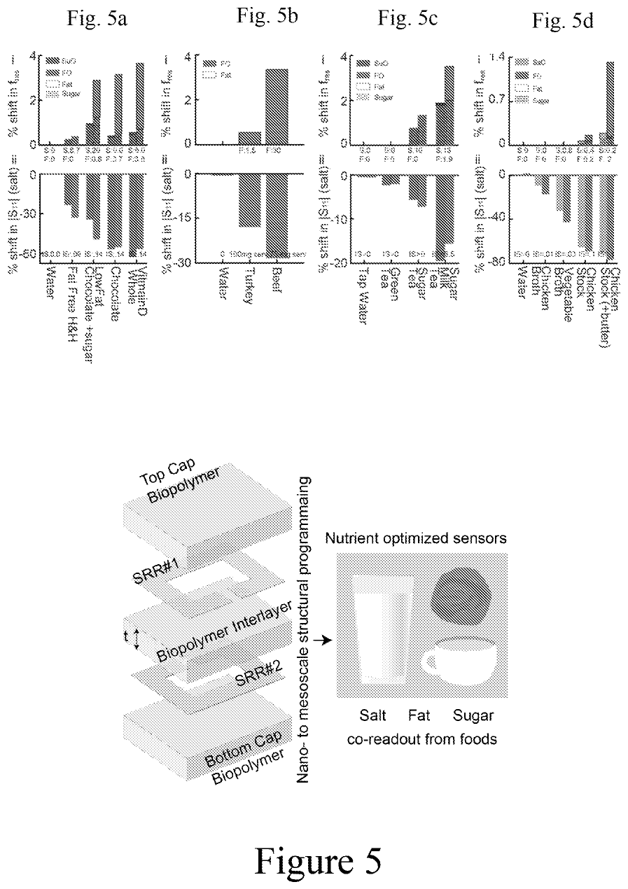

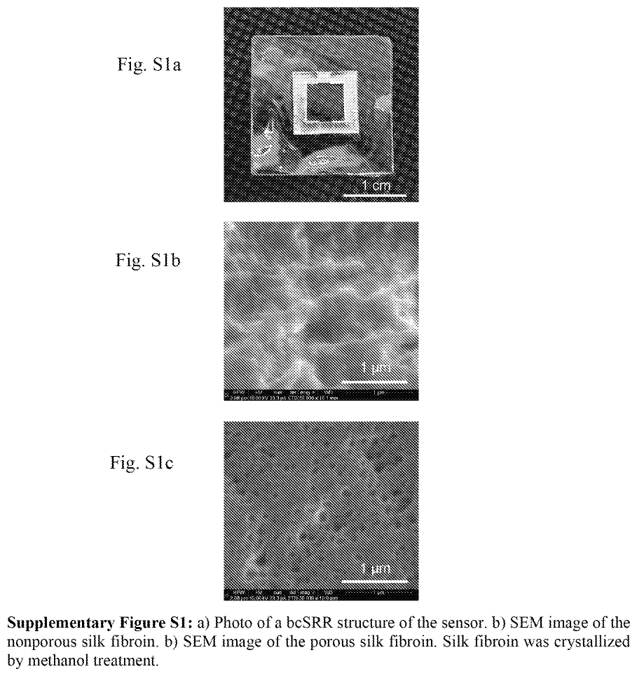

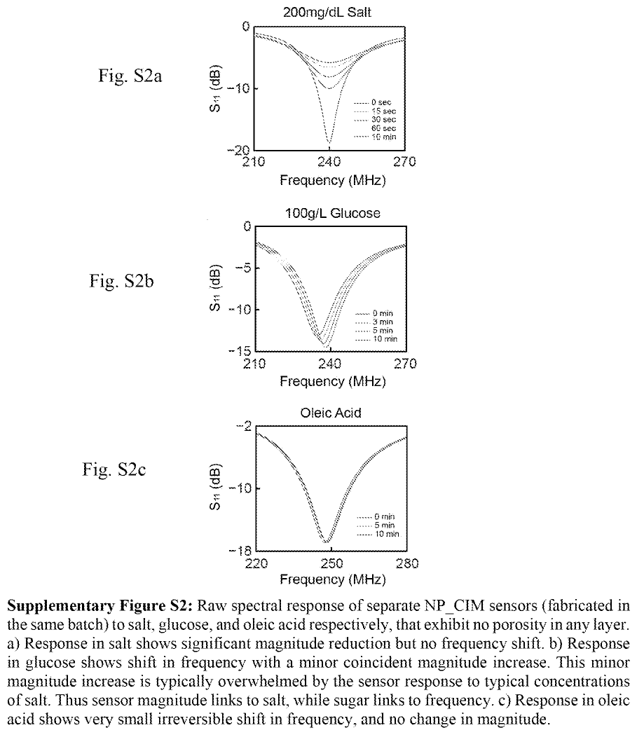

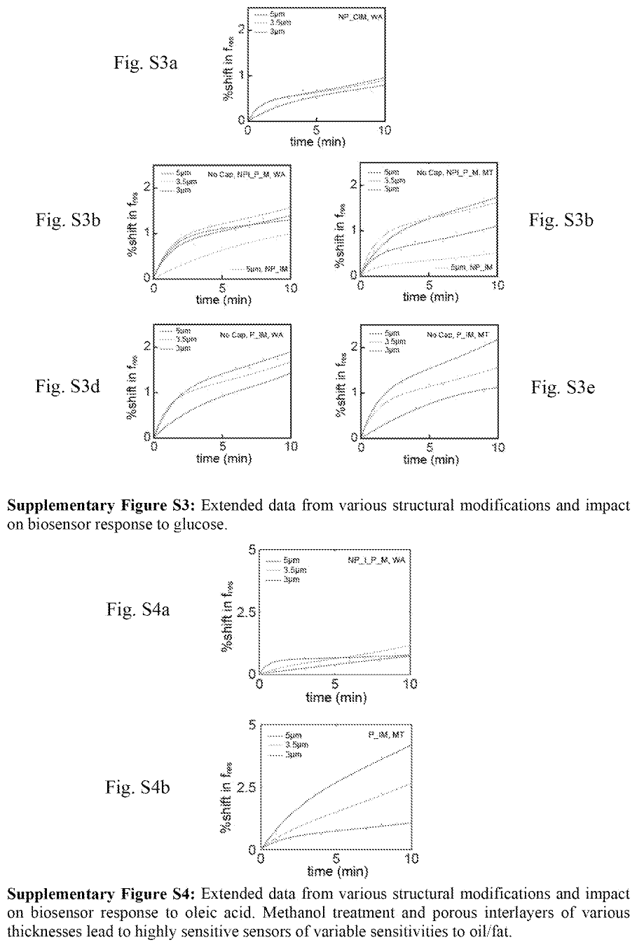

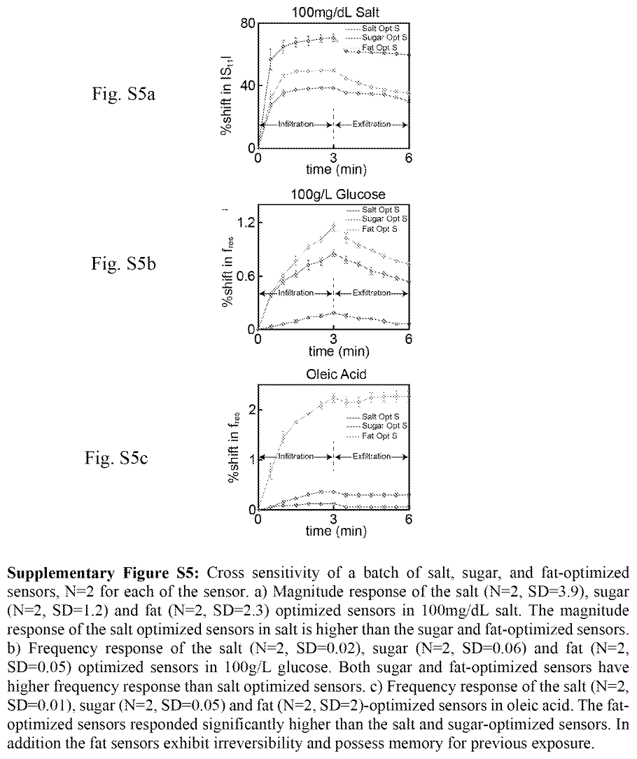

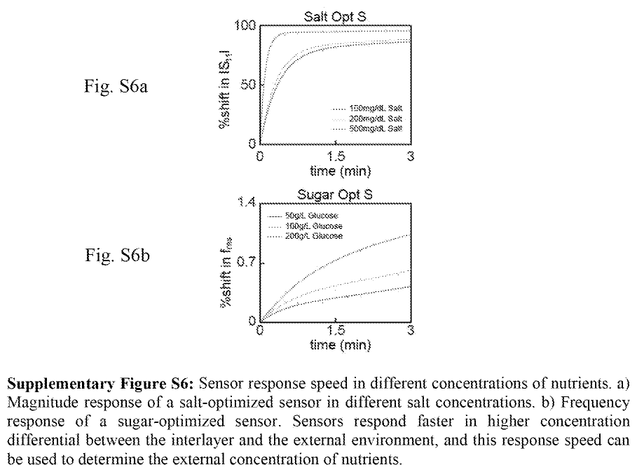

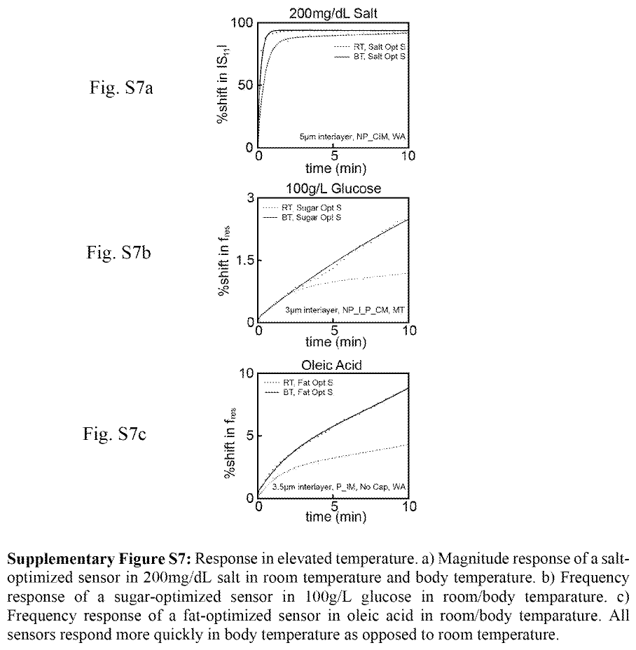

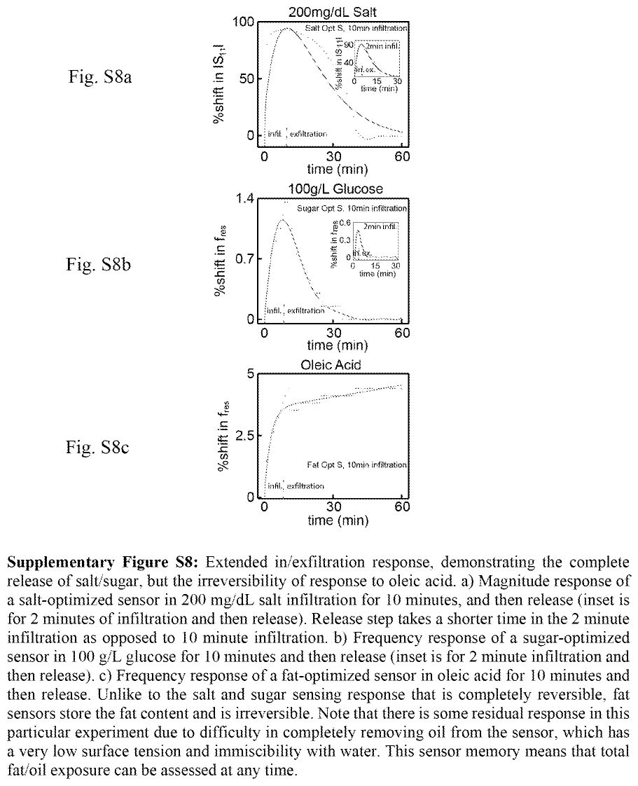

Lastly tested was the ability of the sensors to decouple the nutrient content directly from real foods (FIG. 5). This is relevant because real foods contain many molecules, and it was desired to see how the sensor response correlates to the measurement of real salts/sugars/oils within various foods. Organized was an experiment to study whether the response of a duo of a SaO or SuO sensor alongside a FO sensor could decouple nutrient concentrations in real foods that appear equivalent visually. Shown are four representative studies on solid and liquid foods including: milks of varying nutrient composition, ground meat of varying ratios of protein to fat, tea that has been modulated with additives, and broths/soups of varying concentration of salts and fats. Various foods of modulating nutrition are presented to the sensors, and the sensor response was assessed at 5 minute intervals (note that this type of readout will not be fully accurate because sensors are not completely saturated in response, however one should be able to glean meaningful information on how sensors interact with different foods/nutrients). For these experiments sugar and fat signals are seen in the frequency shift, while salt signals are seen in the magnitude shift.

First assessed was whether SuO alongside FO sensors could assess variations in nutrients present in milk. Shown are the interval response of sensors to milks of varying nutrient content in FIG. 5a (labelled in the figure, and are extracted from the nutritional label of foods). Sugar signals correlated well with the total sugar of various milks, with the sugar-added chocolate milk exhibiting proportionally larger response in the SuO sensor (this simulates cereal milk for example). Zero fat milk presented no extra response in the FO sensor beyond its sugar response, however generated a large extra frequency shift with low-fat chocolate milk, and subsequent saturating signals in the presence of whole chocolate and whole vitamin-d foritified milk (this indicates the positive detection of fat in these liquids). Lower electrolyte signal was seen when immersing sensors in the unfortified H&H, however larger electrolyte signals were seen in fortified formats of milk that exhibit a higher ionic strength. Tested was a SaO and FO sensor with various soups and broths. Magnitude response of the SaO sensor tracked with low salt levels in simple chicken and vegetable broths (IS=0.01 and 0.03 M respectively), while exhibiting larger responses in salty chicken stocks. Broths with low simple sugar concentrations exhibited effectively no frequency shift the SaO sensor, while small and subsequently large excess fat signals appeared in the FO sensor in response to low-fat chicken stock and chicken stock+butter soups respectively. In a solid food test, a FO sensor exhibited proportionally higher fat response to ground beef as opposed to lean turkey meat. Expected results followed for tea that was either unadulterated, modified with sugar, or modified with sugar and milk. Broadly these results indicate that our sensor set can monitor critical nutritional content (simple carbohydrates, oil/fats, and salt) direct from real food irrespective of origin, and that in particular these sensors hone in on major nutrient categories present within real food (sugar, fat, salts). To our knowledge this multi-nutrient co-monitoring direct from real foods (without any sample preparation) is the first of its kind.

### CONCLUSION

The present disclosure provides novel biosensors for the discrimination and co-readout of nutrients from food. This was achieved through the multi-scale engineering of silk biopolymer-interlayer sensors. By manipulating the nano- to meso-structural properties of these multi-layer constructs, obtained are biosensors optimized for salts, simple sugars, and oil/fat. Importantly, this approach uses no delicate nanomaterials or biomolecules, and it is anticipated that such concepts in structural engineering can be adapted to other selective or partially-selective sensors. Uniquely, it was demonstrated that these sensors can discriminate and co-readout nutrients direct from read foods that appear equivalent to the naked eye. It is anticipated that these sensors can be utilized in emerging dietary tools for applications across food tracking and human health.

### EXPERIMENTAL

Chemicals: Bombyx mori silk cocoons are purchased from Mulberry Farms (USA). Sodium Chloride (NaCl), Sodium bicarbonate (Na2CO3), Lithium Bromide (LiBr), D-Glucose, Oleic Acid, Polyethylyne Glycol (PEG-300) are purchased from Sigma Aldrich (USA). All chemicals are used without further modification.

Silk Fibroin Film Synthesis: Silk Fibroin Stock Solution Preparation— Cocoons were boiled in 0.02M Na2CO3 aquaous solution for 30 minutes to extract the sericin proteins, and then dissolve in 20%(w/v) 9.3M LiBr solution in the oven at 60° C. for four hours. The resulting solution was dialysed over 48 hours and finally cetrifuged for 20 minutes to remove any impurities [42].

Porous Silk Fibroin Stock Solution Preparation— Silk fibroin stock solution and PEG-300 (15% weight ratio) were mixed in a controlled way so that the silk does not crystallize during the mixing. After forming the thin film the PE-G300 was released from the film in DI water in a stirred beaker [44].

Silk film Preparation— Nonporous (silk fibroin)/porous (silk fibroin and PEG-300 mixture) silk films were prepared in two different ways. In the water-annealed method, the film was dried overnight followed by water-annealling for four hours in a desiccator at high vacuum (23 psi). In methanol treatment method, the film was dried followed by submersion in a 90% methanol for 10 min and subsequent drying for 4 hours.

Fabrication: Antenna Design— Metal electrode patterns were designed using 2D design tools (such as Layout Editor), and an electronic cutter (Silhouette Cameo 3) was used to create antenna patterns (.e. split rings) by cutting a conductive layer (aluminum or titanium foil) pasted on vinyl film. The negative pattern of the metal features was removed via a tweezer.

Substrate Preparation— A known amount of silk fibroin stock solution corresponding to particular thickness was deposited above of the metal pattern, dried over 4 hours, and water annealed for overnight. Constructs were subsequently re-scaffolded on water-soluble tape, followed by acetone etching of the vinyl layer for the encapsulated version. For constructs without a top capping structure, 10 μm silk was deposited, dried on the bottom metal electrode and water annealed for overnight, followed by rescaffolding on water-soluble tape and acetone etching of the vinyl layer. The top metal layer is kept attached on vinyl.

Interlayer Formation— Another layer of silk fibroin was spun at different speeds onto the exposed side of the metal pattern to form a thin film. Separate films were then exposed to high humidity, and subsequently aligned and embossed together using a custom embossing setup (100° C., 20 psi, 30 seconds) following a water annealing/methanol treatment to form a final, self stable structure composed of silk film-split ring trilayer flanked by water-soluble tape. Before studies, these structures were released from this tape and pre-treated over 3 days in deionized water.

In-Vitro Validation: Resonant response of 1 cm×1 cm sensors were characterized with a Keysight E5063A network analyzer using a 1.5 cm RF Explorer H-loop antenna attached to the coaxial input. For the sensor response to salt, glucose, and fat, 200 mg/dL NaCl, 100 g/L D-Glucose, and Oleic Acid were infiltrated into the sensor and the S11 spectral response was recorded. The the magnitude and resonant frequency were extracted from the S11 plot.

After initial characterization, three designs were used for final optimized sensors. For SaO sensor, water-annealed silk fibroin possessing all nonporous layers along with a 5 μm interlayer is used. For SuO sensors, methanol treated silk fibroin with porous capping and metal layers but a 3 μm non porous interlayer is used. For FO sensor, a 3.5 μm interlayer, water-annealed uncapped construct with all layers being porous is used.

For the step salt response, (50, 100, 200, 300, 400) mg/dL NaCl was added to a SaO sensor and data were taken after 5 minutes. For the step glucose response, 50 g/L glucose was added to a SuO sensor in each increment and data was taken after 10 minutes. For the step fat response, 5 L oleic acid was added to a FO sensor in each increment and data was taken after 5 minutes.

For the temporal response, a SaO sensor was tested in 100 mg/dL NaCl solution first and then reset in DI water before testing in 200 mg/dL NaCl solution. The sensor is then reset in DI water again before testing for the next concentration (500 mg/dL) of NaCl solution. A similar procedure was followed in testing the SuO sensor in 50, 100, and 200 g/L D-Glucose solutions.

For the reversibility response, a SaO, SuO and FO sensor were infiltrated in 200 mg/dL NaCl, 100 g/L D-Glucose, and Oleic Acid respectively for 10 minutes and then released in DI water. The SaO and SuO sensors were also tested for 2 minutes infiltration in respective solutions and then released. For duplicate studies, two of each SaO, SuO and FO sensors from the same batch were tested in 100 g/L D-Glucose, 100 mg/dL NaCl, and Oleic Acid respectively. The data were recorded for 3 minute infiltration and 3 minute exfiltration response, and reset completely in DI for overnight before testing in the next solution.

For body temperature response, temperature was set to 38° C. degree Celsius by a programmable feedback thermometer hotplate. SaO, SuO and FO sensor were tested in 200 mg/dL NaCl, 100 g/L D-Glucose, and Oleic Acid solution respectively to record the temporal response.

For salt and glucose mixture response, a SaO and a SuO sensor were used. A mixture of 100 mg/dL salt and 100 g/L glucose was added to the solution at 38° C. and data was taken continuously. For salt and fat mixture response, a SaO and a FO sensor were used. 100 mg/dL salt was added to the solution at 38° C. for 2 minutes and data was taken continuously. After 2 minutes, salt solution was removed and 10 μL oleic acid was added onto the sensors for 8 minutes while recording data continuously.

Whole food sensor validation: For real food experiments, all foods were purchased from local grocery stores. The final optimized sensors were exposed to the food for 5 minutes and then data was recorded. A SuO and FO sensor were tested in four different types of milk (fat-free, low fat chocolate with 100 g/L of added sugar, chocolate, and Vitamin D enriched whole milk)—these each possess differing nutrient content. A SuO and a FO sensor was tested in green tea in three different conditions: unmodified green tea, 100 g/L sugar added to green tea, and sugar-added milk is added to the sugar green tea forming 50% sugar-milk and 50% sugar-green tea. A SaO and a FO sensor were tested in various soups/broths (unsalted chicken broth, vegetable broth, chicken stock, 20% butter added chicken stock) with differing amounts of salt and fat content. A FO sensor was tested in meat (extra lean ground turkey, fatty beef) for differing fat content.

The documents referred to in the above disclosure by numerals with brackets [ ] are incorporated by reference in the prior application to which this application claims priority.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

What is claimed is:

1. A device comprising multi-scale engineering of silk biopolymer-interlayer sensors for co-monitoring of nutrients.

2. The device of claim 1 wherein, by manipulating various nano- to meso-structural properties of such biosensors, sensors with programmable sensitivity and selectivity to salts, sugars, and oils/fats are obtained.

3. The device of claim 1 configured into a programmable biosensor for wireless readout, wherein characteristics of these passive, wireless nutrient monitors are studied in-vitro.

4. The device of claim 1 wherein the base sensing element is composed of engineered silk fibroin biopolymer interceding two metal electrodes.

5. The device of claim 4, wherein the biopolymer interlayer acts as a lossy dielectric material and can be represented as a parallel combination of a capacitor and a resistor.

6. The device of claim 5, wherein the capacitance and resistance is configured to vary with the changing physical and dielectric properties of the interlayer in response to nutrients.

7. The device of claim 4, wherein open pores are selectively introduced into the silk biopolymer to modulate its selectivity.

8. The device of claim 7, wherein the biopolymer interlayer is composed of a solid phase (hygroscopic, water-pinning silk fibroin), and open phase (or pores within the solid protein film).

9. The device of claim 8, wherein the solid phase interacts only with salt and sugar due to the diffusion of these molecules through the water-carrying silk fibroin.

10. The device of claim 9, wherein for salt, the solid phase will absorb the charged ions and the dielectric will exhibit a large change in the in ε″ or loss of the interlayer.

11. The device of claim 9, wherein for sugars, the solid phase will absorb simple carbohydrates, which induces swelling in the protein, thereby changing the thickness, t.

12. The device of claim 11, wherein the carbohydrates are small-chained such as lactose/sucrose/fructose/glucose.

13. The device of claim 9, wherein the salt and the sugars affect either the real (sugar) or imaginary (salt) portion of the capacitance, and thus each nutrient class can be discriminated from each other readily during measurement.

14. The device of claim 9, wherein the porous section irreversibly absorbs fat from the environment.

15. The device of claim 14, wherein the holes fill with oil/fat over time, replacing water.

16. The device of claim 14, wherein the absorption of the fat is reflected in an irreversible continual shift in ε′ of the sensor when exposed to oil/fat, because oil/fat has a significantly lower permittivity than water.

17. The device of claim 9, wherein in the base unmodified sensor, there are very few pores in the spun-coat biopolymer.

18. The device of claim 17, wherein the base unmodified sensor presents very low sensitivity to fatty acids and almost no sensitivity to triglycerides.

19. The device of claim 9, wherein the sensor is built into a broadside-coupled split ring resonator (bcSRR) format.

20. The device of claim 19, wherein the sensor is interrogated wirelessly and the fundamental response of this wireless sensor can be modelled using a simple series RLC equivalent circuit.
